(19) United States
(12) Patent Application Publication
(10) Pub. No.: US 2017/0151100 A1
(43) Pub. Date: Jun. 1, 2017

(54) Inner Liner Device

(71) Applicant: Suzette Doss, JR., Los Angeles, CA (US)

(72) Inventor: Suzette Doss, JR., Los Angeles, CA (US)

(21) Appl. No.: 15/362,794

(22) Filed: Nov. 28, 2016

Related U.S. Application Data
(60) Provisional application No. 62/260,408, filed on Nov. 27, 2015.

(51) Int. Cl. A61F 13/20; A61F 13/84

(52) U.S. Cl. CPC A61F 13/2022; A61F 13/8405; A61F 2013/8408

(57) ABSTRACT

An inner liner device for battling wetness and odors of feminine discharges from a vagina is provided. The inner liner device comprises a feminine pad insertable into the vagina. The feminine pad is constructed from an absorbent material and provides optimal protection for a fresh, clean feeling.

### CLAIM OF PRIORITY

This patent application claims priority under 35 USC 119(e) (1) from U.S. Provisional Patent Application Ser. No. 62/260,408 filed Nov. 27, 2015, of common inventorship herewith entitled, “Inner Liner,” which is incorporated herein by reference as though the same were set forth in its entirety.

### FIELD OF THE INVENTION

The present invention pertains to the field of personal hygiene, and more specifically to the field of feminine sanitary pads.

### BACKGROUND OF THE INVENTION

Proper hygiene is a key factor in maintaining a healthy and happy existence. Hygiene rituals reduce germs and bacteria and are important for a person to feel clean and fresh on a daily basis. Brushing one s teeth taking a quick shower, or enjoying a long soak in the bathtub are all activities that cleanse and refresh one's body and spirit. Although these habits provide an efficient means of cleaning the body and battling odors, trying to maintain that level of cleanliness throughout a day is very challenging.

Optimal freshness is particularly challenging for females. A monthly period or occasional vaginal discharge due to maladies such as yeast or urinary tract infections and symptoms of menopause leave a female feeling uncomfortable and self-conscious as these discharges typically are accompanied by an unpleasant odor. Many females attempt to solve this problem by utilizing sanitary douches which is impractical to do on a daily basis.

The prior art has put forth several designs for feminine sanitary pads. Among these are:

US Patent Publication 2002/0142693 to Herve Buzot describes a catamenial absorbent device for insertion into a vaginal cavity having two portions, an overwrap containing an absorbent structure. The overwrap has a lower, fluid impermeable barrier, an upper fluid permeable overwrap, and the absorbent structure contains absorbent material therein. The two portions of the overwrap are made from dissimilar materials which are joined at a junction.

U.S. Pat. No. 3,683,912 to Virginia A Olson and Bruce A. Townsend describes an absorbent tampon having a fluid pervious layer of polypropylene fibers disposed on its outer surface. Insertion force for such tampons is reduced more than fifty percent when compared with the insertion force required for conventional tampons.

U.S. Pat. No. 3,508,548 to Josef Hochstrasser and Bernd Messing describes a catamenial tampon which has an optimum capacity for radial expansion and wherein at the same time, independently of the weight of quantity of material used, there is afforded an optimum specific absorption capacity. The tampon comprises a cylindrical fiber form object made up of a fleece of randomly disposed fibers having a specific wet length, at a leading end thereof, of approximately five tenths to one and seven tenths centimeters per gram. The specific wet length is measured by allowing the tampon to expand in a liquid medium in a shallow dish. The fleece is coiled upon itself in a direction normal to the longitudinal axis of the tampon. The tampon contains zones of greater and lesser material concentration distributed at least over the width thereof.

None of these prior art references describe the present invention.

### SUMMARY OF THE INVENTION

It is an object of the present invention to provide a specially constructed hygiene pad configured specifically for battling wetness and odors of feminine discharges.

The present invention is an inner liner device for battling wetness and odors of feminine discharges from a vagina. The inner liner device comprises a feminine pad insertable into the vagina. The feminine pad is constructed from an absorbent material and provides optimal protection for a fresh, clean feeling.

The present invention is a method for battling wetness and odors of feminine discharges from a vagina. The method comprises providing a feminine pad, constructing the feminine pad from an absorbent material, inserting the feminine pad into the vagina, and providing optimal protection for a fresh, clean feeling.

The present invention further includes an inner liner device for battling wetness and odors of feminine discharges from a vagina. The inner liner device comprises a square shaped feminine pad insertable into the vagina wherein the feminine pad is constructed from a cushiony, non-allergenic, absorbent material providing optimal protection for a fresh, clean feeling. The feminine pad absorbs unpleasant residue and odors at the source of the discharge.

### BRIEF DESCRIPTION OF THE DRAWINGS

FIG. 1 is a perspective view illustrating an inner liner device, constructed in accordance with the present invention, showing the inner liner device which absorbs moisture.

FIG. 2 is a perspective view illustrating the inner liner device, constructed in accordance with the present invention, with the inner liner device inserted directly inside a female's vagina to absorb discharge at a discharge source.

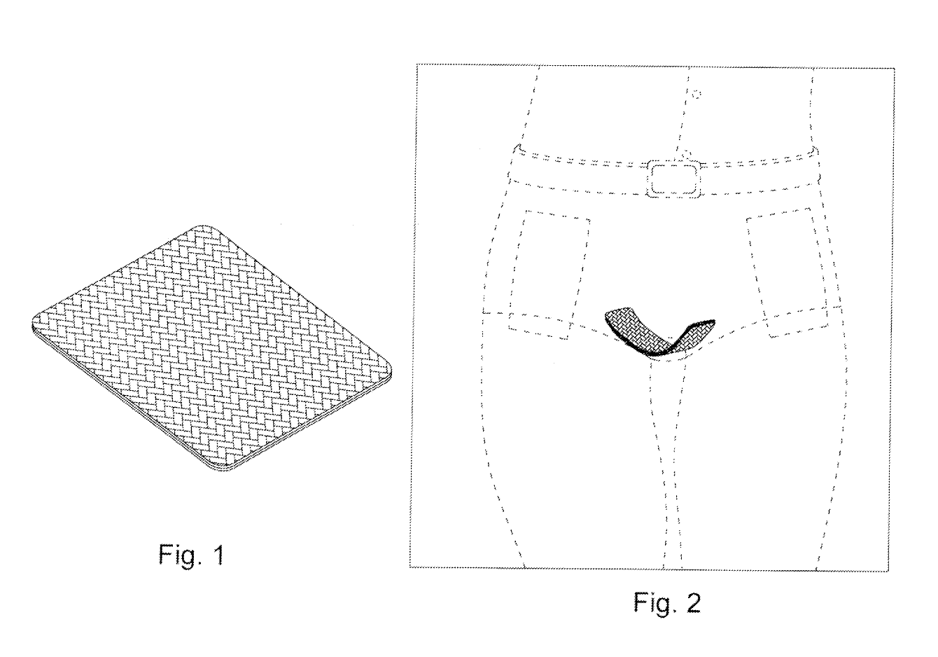

### DETAILED DESCRIPTION OF THE INVENTION

The present invention, hereinafter referred to as an Inner Liner Device, indicated generally at 10, is a specially constructed hygiene pad 12 configured specifically for battling wetness and odors of feminine discharges. The Inner Liner Device 10 provides females with optimal protection for these feminine conditions, enabling them to have a fresh, clean feeling at all times,

The Inner Liner Device 10 of the present invention is a feminine pad 12 that is placed directly inside the vaginal area where it is able to absorb unpleasant residue and smells at the source of the discharge. The feminine pad 12 of the Inner Liner Device 10 is preferably a thin, comfortable, cushiony, and non-allergenic pad. The feminine pad 12 is preferably square in shape allowing the user to easily manipulate the feminine pad 12 although having the feminine pad 12 be a different shape including, but not limited to, round, triangular, rectangular, oval, etc., is within the scope of the present invention. The feminine pad 12 can be constructed as unscented or scented with a fragrance component available in a variety of scents to appeal to individual tastes.

The Inner Liner Device 10 of the present invention can be packaged and sold in compact cartons of several individually wrapped units per container. The Inner Liner Device 10 can also be distributed individually via restroom vending machines.

Use and application of the Inner Liner Device 10 of the present invention is simple and straightforward. A user inserts the feminine pad 12 of the Inner Liner Device 10 into her vagina prior to or following a menstrual cycle or while experiencing menopausal symptoms, the most common causes of odoriferous vaginal discharge. A user can easily carry one or more feminine pads 12 in her purse, backpack, or briefcase when leaving home. Practical and safe, the Inner Liner Device 10 works directly from the emanating source to eliminate internal and external residue which causes discomfort, providing a hygienic means of maintaining freshness.

Safe to use at any time, the Inner Liner Device 10 of the present invention is particularly useful before and after a woman's normal monthly period or following sexual activity. More than a mere pad, the Inner Liner Device 10 also thwarts a spread of germs and bacteria, thus ensuring optimal feminine health. Able to absorb any unpleasant discharge and smells on contact, then release an aromatic loud of fragrance, the inner Liner Device 10 promotes cleanliness, convenience and confidence for women of all ages.

Although this invention has been described with respect to specific embodiments, it is not intended to be limited thereto and various modifications which will become apparent to the person of ordinary skill in the art are intended to fall within the spirit and scope of the invention as described herein taken in conjunction with the accompanying drawings and the appended claims.

1. An inner liner device for battling wetness and odors of feminine discharges from a vagina, the inner liner device comprising:
a feminine pad insertable into the vagina;
wherein the feminine pad is constructed from an absorbent material;
wherein the feminine pad provides optimal protection for a fresh, clean feeling.

2. The inner liner device of claim 1 wherein the feminine pad absorbs unpleasant residue and odors at the source of the discharge.

3. The inner liner device of claim 1 wherein the feminine pad is constructed from a cushiony, non-allergenic material,

4. The inner liner device of claim 1 wherein the feminine pad has a square shape.

5. The inner liner device of claim 1 wherein the feminine pad is unscented.

6. The inner liner device of claim 1 wherein the feminine pad is scented.

7. A method for battling wetness and odors of feminine discharges from a vagina, the method comprising:
providing a feminine pad;
constructing the feminine pad from an absorbent material;
inserting the feminine pad into the vagina; and
providing optimal protection for a fresh, clean feeling.

8. An inner liner device for battling wetness and odors of feminine discharges from a vagina, the inner liner device comprising:
a square shaped feminine pad insertable into the vagina;
wherein the feminine pad is constructed from an cushiony, non-allergenic, absorbent material;
wherein the feminine pad provides optimal protection for a fresh, clean feeling:
wherein the feminine pad absorbs unpleasant residue and odors at the source of the discharge.

9. The inner liner device of claim 8 wherein the feminine pad is unscented.

10. The inner liner device of claim 8 wherein the feminine pad is scented.
